(19) United States
(12) Patent Application Publication
(10) Pub. No.: US 2003/0135171 A1
(43) Pub. Date: Jul. 17, 2003

(54) Sleeve having integral and permanent therapeutic materials for application of a broad range of therapeutic modalities

(76) Inventors: Aaron Neil Ingram, Canton, GA (US); Mark Herman Bruder, Alpharetta, GA (US)

Correspondence Address: Aaron N. Ingram, 3150 Engineering Parkway, Alpharetta, GA, 30004, US

(21) Appl. No.: 10/341,806

(22) Filed: Jan. 14, 2003

Related U.S. Application Data
(60) Provisional application No. 60/349,335, filed on Jan. 14, 2002.

(51) Int. Cl. A61F 13/00

(52) U.S. Cl. 604/308

(57) ABSTRACT

The invention provides a sleeve containing a non-removable therapeutic agent within that is used to provide a variety of therapeutic modalities. The sleeve is constructed of an inner and outer layer of flexible materials suitable to pulled onto a portion of the body such as elbow, wrist, ankle, knee, or other limb for application of therapy. A therapeutic agent of choice becomes non-removable, permanently contained between the inner and outer layers. Various therapeutic agents may be chosen to delivery a wide range of modality. Microwave activated particulates may be contained within the inner and outer coverings to provide moist heat therapy. Metallic particulates may be chosen for cold therapy applications. Outer layers may be insulated to improve heat retention to extend therapeutic time. So constructed, a therapeutic sleeve is provided to delivery heat, cold, or contrast therapy to an affected body part of a human or animal subject.

### CROSS REFERENCE TO RELATED APPLICATIONS

This application claims priority to, and the benefit of the filing date of U.S. Patent Application No. 60/349,335, filed on Jan. 14, 2002, in the United states Patent and Trademark Office.

### FIELD OF THE INVENTION

The present invention relates in general to application of a wide range of therapeutic modalities (heat therapy, cold therapy, contrast therapy). More particularly, the present invention relates to a sleeve used to provide these modalities.

### BACKGROUND OF THE INVENTION

Application of heat therapy, cold therapy (cryotherapy), and contrast therapy (alternating heat and cold therapy) is used to treat a multitude of conditions including muscle and joint pain, strains, sprains, a variety of arthritic conditions, menstrual cramps, and a variety of other maladies. The earliest applications of heat therapy used hot towels or hot stones wrapped in towels. Electric heating pads, clay filled packs kept in hot water tanks, gel packs, alumina and grain packs heated in the microwaves, hot water bottles, water recirculation systems, and paraffin baths have been the methods available to application of heat therapy. Moist heat therapy is the preferred method in most circumstances, since it provides for deeper penetration of heat at lower temperatures. Some of the aforementioned systems can provide moist heat, but some, such as hot water bottles, gel packs, and paraffin baths cannot. Application of cold therapy has been done using ice bags, gel packs, and water recirculation systems. In each of the methods of application, a heat or cold pack is directly applied to the body for the treatment time of typically 20 to 30 minutes. In some cases, ties or elastic straps have been provided to affix the treatment pack to the body, but in many cases the pack simply rests on the targeted area. Straps and ties are cumbersome and are often difficult to self-administer. Gel packs require heating in the microwave and then placing inside a cover which is then applied to the affected body part.

A growing strategy for treatment of many joint pains involves application of heat, cold, or contrast therapy in conjunction with exercise or movement therapies. These are often self-administered treatments prescribed by medical professionals or standard protocols. Compliance is a major issue in self-administered treatments. Ease of application is often the single most important factor in the success of home treatment. Effective treatment necessitates products that can provide the modality (hot, cold, or contrast), simultaneously provide consistent contact with the targeted area, allow ease of application and minimal steps in order to insure proper compliance in a self-administered environment.

### SUMMARY OF THE INVENTION

The invention is a sleeve designed for the application of a broad range of therapeutic modalities. Features include integral and permanent therapy materials (therapy source which provide modalities such as heat therapy, cold therapy, contrast therapy, etc.), ease of application and removal in a variety of situations, and the capability of delivering a single or multiple therapeutic modalities simultaneously, concurrently, and/or consecutively. Invention is applicable for both human and animal therapeutic modalities,

### BRIEF DESCRIPTION OF THE DRAWINGS

FIG. 1 is a frontal view of the therapeutic sleeve of the present invention

FIG. 2 is a cross-section of a therapeutic sleeve for single modality

FIG. 3 is a cross-section of a therapeutic sleeve with dual modality

FIG. 4 shows various typical uses on human subject

### DETAILED DESCRIPTION OF THE DRAWINGS

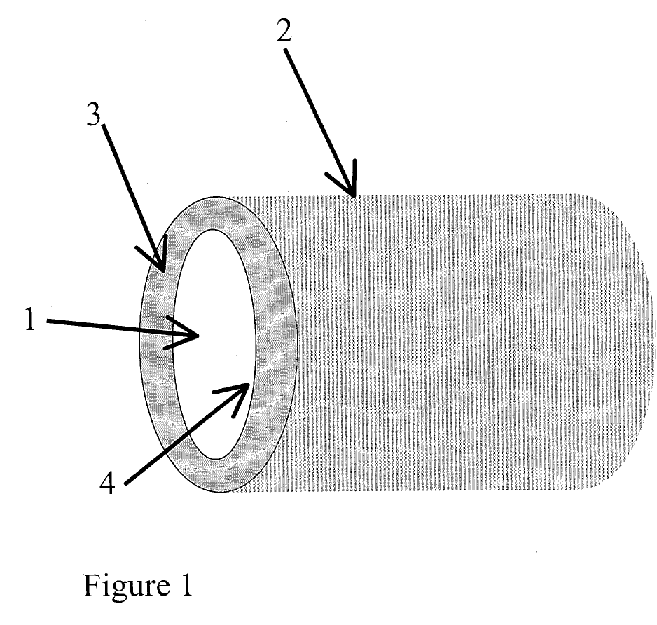

FIG. 1 shows a typical embodiment of the present invention featuring an outer (shell) covering (2) and inner (shell) covering (4) which are joined to form a sleeve and a containment area (3) into which is permanently sealed an intrinsic therapy source for the preferred modality. There is an opening (1) thru which a portion of the body may be placed.

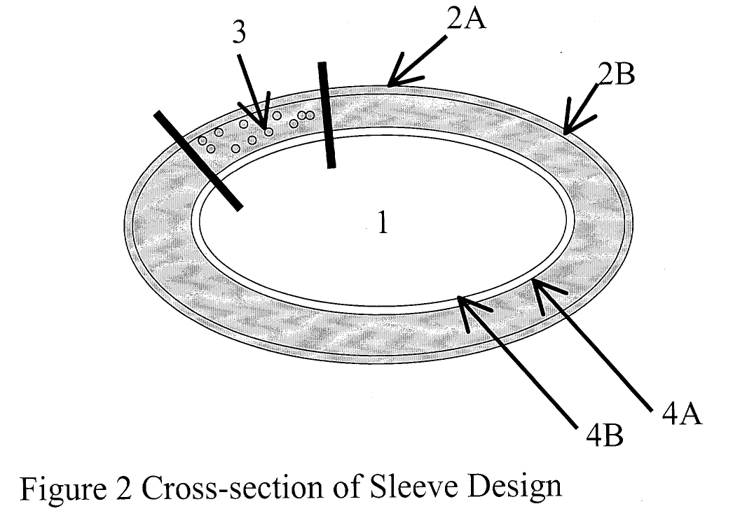

FIG. 2 shows more detail of a typical embodiment in cross-section. (1) is the opening thru which a area of the body can be placed. (2A) and (2B) represent the upper and lower layer of the top containment material. (3) represents the therapeutic material permanently contained within the sleeve. (4A) and (4B) shows the upper and lower layer of the inside containment material.

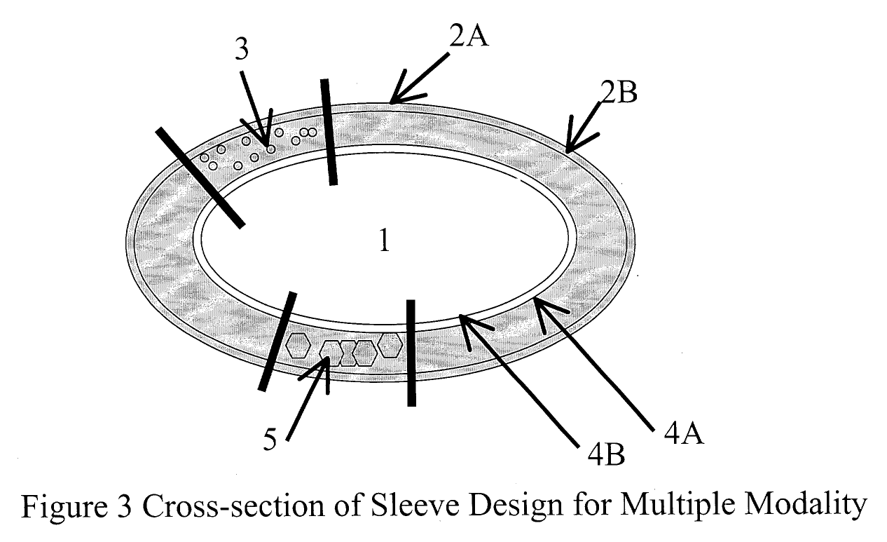

FIG. 3 shows more detail of a typical embodiment in cross-section. (1) is the opening thru which a area of the body can be placed. (2A) and (2B) represent the upper and lower layer of the top containment material. (3) represents the therapeutic material permanently contained within the sleeve. (4A) and (4B) shows the upper and lower layer of the inside containment material. (5) shows the addition of a second therapeutic material permanently sealed in the sleeve.

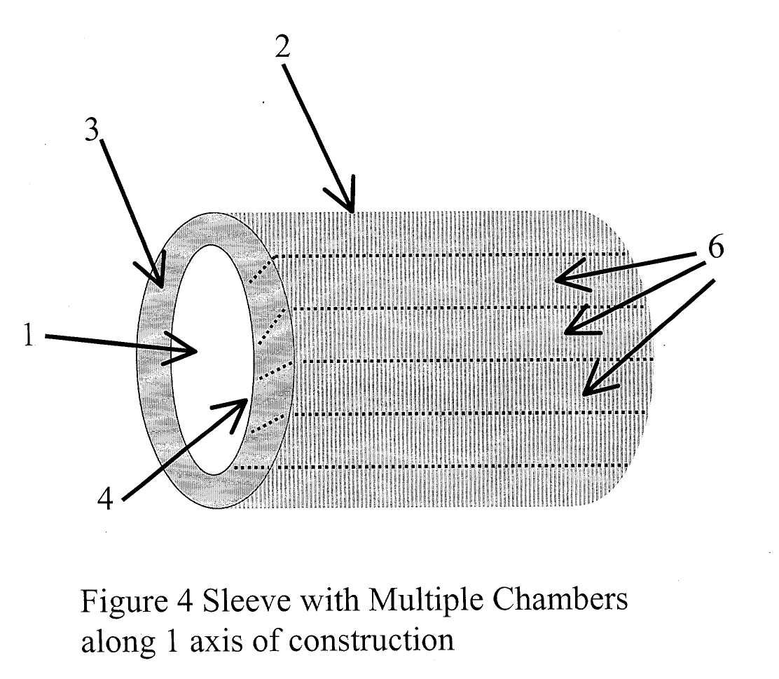

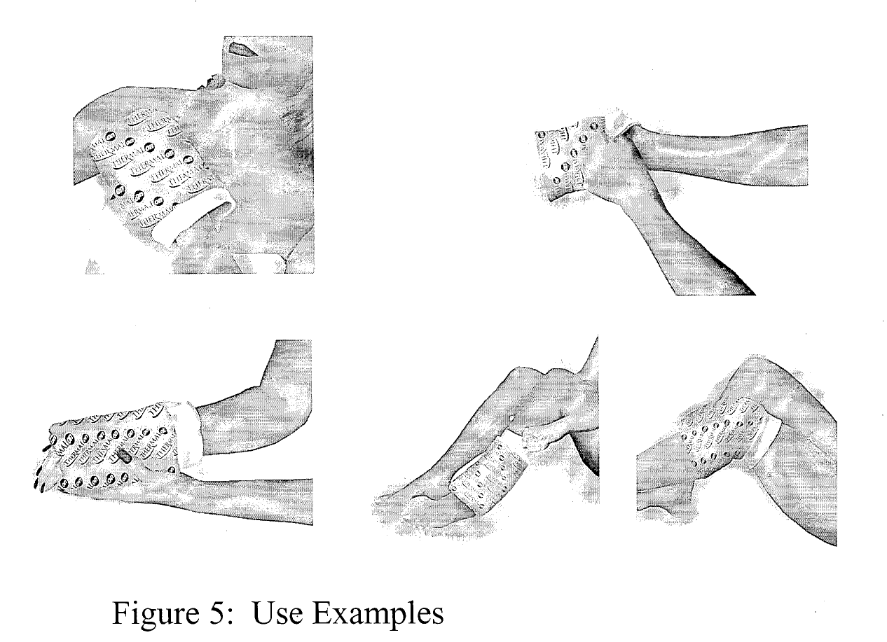

FIG. 4 shows a modification of FIG. 1 which features independent chambers (6) running along one axis in which the therapeutic material (3) is permanently fixed. Independent chambers can also be configured along another axis (not shown).

FIG. 5 shows application of therapeutic sleeve to various body parts using a human subject.

### DETAILS OF CONSTRUCTION

Sleeve Construction

1) Sleeve construction includes a non-removable, permanent, and integral therapeutic material (therapy source).

2) Construction is a sleeve configuration designed to provide easy application of the desired modality to the affected area of a human or animal subject

a. Sleeve size (diameter, length, girth) may vary depending upon anatomical region where therapeutic treatment is needed

b. Sleeve size may be non uniform across its length or girth to provide for proper anatomical placement

3) Materials of construction for the sleeve vary depending upon modality required. Material choices include, but are not limited to, woven fabrics, knitted fabrics, non-woven constructions, elastics, coated materials, laminates, open and closed cell foams, etc.

a. Materials may be hygroscopic

b. Materials may be hydrophilic

c. Materials may be hydrophobic

d. Materials may contain additives/agents to inhibit bacterial or fungal growth

4) Methods of construction include but are not limited to sewing, ultrasonic bonding, RF sealing, adhesive application, lamination, etc.

5) Construction may be of a single material or a combination of materials

6) Construction may vary to provide for appropriate modality.

a. Construction may include a variety of material choices. Examples include but are not limited to the following

i. A non-permeable layer/material

ii. An insulating material or layer

iii. Components to add stretch and/or recovery

iv. A variety of combinations of material choices appropriate for the therapeutic modality chosen

b. Construction may have localized (regionally specific) areas of material selection to enhance the modality being used.

c. Construction may include a multiplicity of above.

Construction may provide for various levels of compression where appropriate for the modality used. Material choices include but are not limited to woven fabrics, knitted fabrics, non-woven constructions, elastics, coated materials, laminates, etc.

d. Compression may be achieved by using materials with elasticity (and/or recovery) alone

e. Compression may be achieved by using material combinations having both elastic and non-elastic characteristics

f. Compression may be achieved by varying the amount of elasticity in the materials chosen

7) Construction can provide for limited or no compression where appropriate for the therapeutic modality chosen.

a. Wound treatment

b. Support

c. Placement of therapy source on specific anatomical location

8) Product may provide comfort and/or support when therapeutic treatment is completed

9) Product may be either reusable or disposable

10) Sleeve may include decorative elements designed to improve appearance or aesthetics.

Non-Removable, Permanent, Therapy Materials (Therapy Source)

11) Non-removable therapy materials (Therapy Source) are integral to the invention and are permanently installed, affixed, or attached within the sleeve.

a. Therapeutic materials (therapy source) may be partially or fully self-contained

b. Therapeutic materials (therapy source) may be contained by the sleeve

c. Therapeutic materials may be naturally occurring or synthetic

d. Examples of therapeutic materials include, but are not limited to, expanded alumina, molecular sieve, silica, naturally occurring and synthetic zeolites, clay, bentonite, as well as several starches, SAP (super absorbing polymers), cellulosics, urethane gels, silicones, various heat conducting fluids, natural grain products, etc.

e. Therapeutic materials may be capable of delivering an antibiotic, analgesic, or entity capable of inhibiting bacterial or fungal growth.

f. Therapeutic materials may be hygroscopic

g. Therapeutic materials may be hydrophilic

h. Therapeutic materials may be hydrophobic

i. Therapeutic materials may contain additives to inhibit biological growth (bacterial, fungal, etc)

j. Therapeutic materials may be aromatic

k. Therapeutic source may be absorbing

l. Therapeutic source may include construction details suitable to maintain its relative position with regard to the entire unit during use

m. Therapeutic source may have channels designed to distribute the therapy appropriately on the application area

n. Therapeutic source may occupy significant surface area of the product or may be limited and localized for application to a specific anatomy

o. Therapeutic source may contain a multiplicity of components chosen for specific modality

Therapeutic Modalities Considered

1) Heat therapy (Moist or Dry)

2) Cold therapy

3) Contrast therapy (Application of Alternating Heat and Cold Therapy)

4) Compression therapy

5) Exercise therapy

6) Massage therapy

7) Wound treatment

8) Topical application of antibiotic or analgesic

9) Support

10) Any combination of Above

Examples of Use

The invention can be used to deliver a variety of therapeutic modalities. Examples of use include, but are not limited to, the following:

1) Application of heat and/or cold therapy to joints of the hands and feet as found in certain arthritic conditions

2) Application of heat therapy, cold therapy and compression therapy as related to (repetitive stress) injury

3) Simultaneous application of heat therapy and exercise therapy for arthritic fingers

4) Application of contrast therapy to muscle strain of the calf

5) Equestrian heat, cold, and compression therapy to leg quarter

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the invention. Moreover, although specific terms are employed herein, they are used only in a generic and descriptive sense, and not for the purposes of limiting the described invention. The words “a”, “an” or “the” can mean one or more, depending upon the context in which the words are used hereinabove.

We claim:

1. An article for use as a sleeve to deliver therapeutic modality, said article comprising a flexible inner and outer layer with a non-removable therapeutic agent (therapy source) permanently contained between said layers.

2. The article of claim 1, wherein the therapeutic modality is heat.

3. The article of claim 1, wherein the therapeutic modality is cold.

4. The article of claim 1, wherein the sleeve is used for multiple modality therapy.

5. The article of claim 1, wherein the backing and shell each comprise a synthetic material.

6. The article of claim 5, wherein the backing and shell material each comprise a group of synthetic fabrics consisting of olefins, polyester, urethane, nylon.

7. The article of claim 1, wherein the backing and shell material each comprise a natural fiber.

8. The article of claim 1, wherein multiple fabrics of construction may be used.

9. The article of claim 1, wherein the therapeutic agent may be a solid.

10. The article of claim 9, wherein the therapeutic agent may be particulate.

11. The article of claim 1, wherein the therapeutic agent may be a fluid.

12. The article of claim 11, wherein the therapeutic agent may be mixture of fluids.

13. The article of claim 11, wherein the therapeutic agent may undergo a phase change during the therapeutic treatment.

14. The article of claim 5, wherein both layers may be permeable to vapor.

15. The article of claim 5, wherein one layer may be impermeable to vapor and liquid.

16. The article of claim 5, wherein one layer may be insulating.

17. The article of claim 1, wherein the therapeutic modality of the sleeve is activated by microwave action.

18. The article of claim 1, wherein the therapeutic modality of the sleeve is activated by freezing.

19. The article of claim 1, wherein the sleeve is comprised of a multiplicity of fabrics comprising the inner and outer layer.

20. The article of claim 1, wherein the inner and outer layer (shell) is comprised of an elastic material which provides compression in conjunction with other modalities.
